(19) United States
(12) Patent Application Publication
(10) Pub. No.: US 2007/0270355 A1
(43) Pub. Date: Nov. 22, 2007

(54) Sport drink containing amino acids and carbohydrates

(76) Inventors: Ramon D. Garcia, Rowlett, TX (US); Lawrence Murphy, Richardson, TX (US); Cesar Pena, Garland, TX (US); Leovares Mendez, Rowlett, TX (US)

Correspondence Address: WINSTEAD PC, P.O. BOX 50784, DALLAS, TX, 75201, US

(21) Appl. No.: 11/801,715

(22) Filed: May 10, 2007

Related U.S. Application Data
(60) Provisional application No. 60/799,545, filed on May 11, 2006.

(51) Int. Cl. A61K 31/70; A61K 31/4172; A61K 31/405; A61K 31/401; A61K 31/198

(52) U.S. Cl. 514/23; 514/400; 514/423; 514/419; 514/561; 514/562; 514/563; 514/564; 514/565; 514/566; 514/567; 514/53; 514/61

(57) ABSTRACT

A composition includes a plurality of amino acids. The plurality of amino acids includes at least one essential amino acid and at least one non-essential amino acid. The plurality of amino acids also includes at least one branch-chain amino acid. The composition also includes a source of carbohydrates. The compositions also includes purified water. The plurality of amino acids comprises about 1 wt % of the composition. A composition includes a plurality of amino acids, sodium citrate, sodium chloride, potassium phosphate, flavoring, a source of carbohydrates, and purified water. In some embodiments of the composition, the plurality of amino acids includes alanine, arginine, aspartate, cystine, glutamine, glycine, histidine, isoleucine, leucine, lysine, methionine, phenylalanine, proline, serine, threonin, tryptophan, tyrosine, and valine.

### CROSS-REFERENCE TO RELATED APPLICATIONS

This application claims the priority benefit under 35 USC § 119(e) to, and incorporates by reference the entire disclosure of, Provisional Patent Application No. 60/799,545, filed May 11, 2006.

### BACKGROUND

1. Technical Field

The present invention relates generally to compositions for use as dietary supplements, and more particularly to a liquid composition for use during and after exercise to provide energy and nutrition for continued exercise and improved recovery time.

2. History of Related Art

Physical activities such as strenuous exercise involve a very complex set of chemical reactions in the body that result in muscle breakdown and depletion of muscular energy reserves. When exercise occurs in moderation, energy that is stored in the muscle groups in the form of glucose is converted into energy for the muscles to use. During this aerobic process, oxygen is consumed from the bloodstream by the muscles and waste is processed away from the muscle groups. During vigorous exercise, which may require a sudden burst of energy, for example, weightlifting or hard running chasing after a baseball, muscles may use up all the available oxygen and glucose faster than the bloodstream can supply them. In this occurrence, the muscles are starved for energy and have to rely on “emergency reserves” called phosphorylcreatine.

In both types of exercise, energy is used and muscle tissues are worked. As muscle tissue is worked, some of the muscle tissues begin to break down at the cellular level, releasing water and proteins that constitute the cell. Additionally, a by-product of the glucose consumption process, lactic acid, builds up in the muscle tissue and creates a state of acidosis, further breaking down muscle tissue. As a result, the muscle tissue becomes damaged, resulting in a “sore” feeling later on. In addition to the depletion of energy reserves, during exercise a person loses electrolytes and minerals essential to metabolic and nervous system processes by sweating and oxidation.

Combining all these effects, a person during exercise may soon become tired as energy reserves are consumed. Later, after ceasing exercise, a person may continue to feel tired, drained, and sluggish for hours after exercising because the body lacks water, minerals, carbohydrates, and proteins necessary not only to replenish its reserves but also repair the damage that exercise has caused to the muscular tissues. Typically a person would consume a variety of foodstuffs that would eventually replenish all of the lost materials and support the body in repairing the damaged muscle tissues.

A composition that helps quickly replace nutrients and chemicals, especially amino acids, lost during strenuous exercise from the muscle tissues and also speeds recovery time by supporting muscle tissue rebuilding is desired.

### SUMMARY OF THE INVENTION

The summary of the invention is not intended to represent each embodiment or every aspect of the present invention.

A composition includes a plurality of amino acids. The plurality of amino acids includes at least one essential amino acid and at least one non-essential amino acid. The plurality of amino acids also includes at least one branch-chain amino acid. The composition also includes a source of carbohydrates. The compositions also includes purified water. The plurality of amino acids comprises about I % by weight (wt%) of the composition.

A composition includes a plurality of amino acids, sodium citrate, sodium chloride, potassium phosphate, flavoring, a source of carbohydrates, and purified water. In some embodiments of the composition, the plurality of amino acids includes alanine, arginine, aspartate, cystine, glutamine, glycine, histidine, isoleucine, leucine, lysine, methionine, phenylalanine, proline, serine, threonin, tryptophan, tyrosine, and valine.

### DETAILED DESCRIPTION OF ILLUSTRATIVE EMBODIMENTS OF THE INVENTION

In some embodiments, a substantially isotonic liquid composition containing sodium, potassium, chlorides, carbohydrates, water, and amino acids. In some embodiments, the composition is designed for consumption during physical exertion to provide an immediate source of carbohydrate energy. In some embodiments, the composition is designed for consumption during physical exercise to provide a source for replacement of water, sodium, and potassium lost during physical exertion. In some embodiments, the composition is designed for consumption during physical exertion to provide an immediate source of amino acids for preventing muscle breakdown and fostering muscle repair. In some embodiments, the composition is designed for consumption during physical exertion to provide an immediate source of BCAA for repairing and strengthening muscles, ligaments, and tendons over a period of time, such as, for example, 24-48 hours after consumption. In some embodiments, the composition is desgined for consumption after physical exertion to support some or all of the aforementioned goals.

In some embodiments, a plurality of amino acids may be included in an aqueous composition for ingestion. In some embodiments, the plurality of amino acids may include amino acids with branched chains. The plurality of amino acids may be from include one or more of non-essential and essential amino acids and combinations thereof.

Amino acids are small molecular units that are used by organic lifeforms to construct more complex molecules, such as proteins, to perform higher cellular biological functions such as growth, repair, and reproduction. Each protein is made up of a unique sequence and number of amino acids, sometimes numbering in the thousands of units for one protein molecule. In humans, proteins can be visibly seen in the form of muscles, tendons, organs, glands, nails, and hair. After water, proteins make up the next portion of the mass of a human being. Amino acids also may also be used as a source of energy by being broken down in the urea and citric acid cycles. Glucogenic amino acids can be converted to glucose through gluconeogenesis.

The twenty standard amino acids, called “proteinogenic” acids, are used by cells for protein biosynthesis. The twenty amino acids may be synthesized from other molecules, but living organisms differ as to which ones they can self-synthesize and which ones must be provided through dietary intake. The ones that cannot be synthesized by an organism are called “essential” amino acids in that they must be acquired by other means, mainly by consuming foods with these amino acids. The ones that can be self-synthesized by an organism from other molecules are called “non-essential” amino acids. There are also amino acids that depend on the developmental state of the organism and whether it can self-synthesize a particular amino acid or not. These amino acids are called “semi-essential” amino acids in that they are essential in one period of life but not another.

In humans, eight amino acids are generally regarded as essential: isolucine, leucine, lysine, methionine, phenylalanine, threonine, tryptophan, and valine. Two others, histidine and arginine, are semi-essential in that only human children (due to their developmental stage) and potentially seniors (due to declining health) can not self-produce these acids due to their stage of metabolic development. Essential and, if required, semi-essential, amino acids must be consumed in foods to supply the necessary biological components for the body to create and use these acids for protein construction and energy. The remaining ten amino acids in normally healthy humans are considered non-essential: alanine, asparagine, aspartate, cysteine, glutamate, glutamine, glycine, proline, serine, and tyrosine.

Of the essential amino acids, leucine, isoleucine, and valine are also referred to as “branched-chain amino acids” (BCAA). This designation is based upon the molecular structure of these three amino acids comparatively with the other 17 amino acids. The BCAAs possess large aliphatic side groups, making these amino acids comparatively more hydrophobic and rigid than the other amino acids. Research has revealed that BCAAs are needed for the maintenance of muscle tissue and appear to preserve muscle stores of glycogen, which is a storage form of carbohydrate that can be converted into energy by the body when the muscle is under stress, such as from exercise. The BCAAs also appear to help prevent muscle protein breakdown during exercise and further stimulate rebuilding and repairing of muscle fibers.

Alanine, or (S)-2-aminopropanoic acid, is a non-essential amino acid formed from reductive amination of pyruvate. It is used in the aniline cycle to synthesize glucose, an important source of energy for the body, from proteins. It is also suspected that alanine protects against the buildup of toxic substances released from muscle proteins broken down during strenuous aerobic exercise. Most protein-rich foods contain alanine.

Arginine, or 2-amino-5-(diaminomethylidene amino)pentanoic acid, is a semi-essential amino acid synthesized in the urea cycle. Arginine plays important roles in cell division, wound healing, ammonia removal, the release of growth and other hormones necessary for the repair of muscles and reduction of fat, and is a component of collagen. Arginine is a precursor of the hormone nitric oxide. Arginine has been found in chocolate, dairy products, nuts, certain grains, and soybeans.

Aspartate, or (2S)-2-aminobutanedioic acid or aspartic acid, is a non-essential amino acid formed from oxaloacetate by transamination. Aspartate is potentially a neutrotransmitter, an excitotoxin, and is a metabolite in the urea cycle. Aspartate is suspected to increase stamina and metabolism and assist minerals in traversing the intestinal lining/blood vessel interface during digestion.

Cysteine, or (2R)-2-amino-3-sulfanyl-propanoic acid, is a non-essential amino acid that is often found in its more stable and oxidized diametric form cystine, which is two cysteine residues joined by a disulfide bond. Due to this ability to undergo redox reactions, cysteine has antioxidant properties and is also an important source of sulfur in human metabolism. Cysteine is the rate-limiting component in the synthesis of glutathione, an important antioxidant, and is incorporated into insulin where it acts to stabilize the hormone against denaturization. Cysteine readily binds to heavy metal ions. Cystine supports the burning of fat and building of muscles during exercise. Hair and skin is made up of a significant amount of cystine.

Glutamine, or (2S)-2-amino-4-carbamoyl-butanoic acid, is a non-essential amino acid that is the most prevalent amino acid in muscles, where it helps to maintain and build muscle tissues. Glutamine also serves an important function as a nitrogen receiver and donor in the nitrogen metabolism. Glutamine supplements are used in bodybuilding and exercise as well as a means to relieve cramping and pain in seniors. Glutamine has also been shown to improve intestinal and gut-barrier function, and it is used as a supplement after abdominal surgery to aid recovery time.

Glycine, or aminoethanoic acid, is a non-essential amino acid that is the simplest in structure since it has no side-chains—all other amino acids are structural derivatives of glycine. Glycine therefore acts as the most flexible and bendable joint in a protein chain. Glycine is an inhibitory neurotransmitter in the central nervous system, especially in the spinal cord, the brainstem, and the retina. Glycine acts to improve glycogen storage and helps to repair damaged tissues in the body.

Histidine, or 2-amino-3-(3H-imidazol-4-yl)propanoic acid, is a semi-essential amino acid in that human children and some seniors cannot self-synthesize histidine due to metabolic developments. Histidine is found in abundance in hemoglobin, is important in the maintenance of myelin sheaths (the protective layer around nerve cells), and is necessary for the production of both red and white blood cells. Histidine is found naturally in fruits, meats, milk products, and green vegetables.

Isoleucine, or (2S,3S)-2-amino-3-methylpentanoic acid, is an essential amino acid that is also a branched-chain amino acid. Isoleucine acts in the formation of hemoglobin, has been found to regulate blood sugars and energy levels, and is involved in healing and repair of muscle tissue, skin, and bones. Sources of isoleucine include eggs, soybeans, milk products, and cereal grains.

Leucine, or (S)-2-amino-4-methyl-pentanoic acid, is an essential amino acid, a BCAA, and is also an isomer of isoleucine. Leucine is the most common amino acid found in protein chains. Leucine is essential in adults for maintaining nitrogen equilibrium and plays a part in the maintenance of muscle by regulating the synthesis and breakdown of muscle tissue. Leucine may also regulate the production of growth hormone.

Lysine, or (S)-2,6-diaminohexanoic acid, is an essential amino acid that plays a major role in the absorption of calcium in the body. Lysine is also involved in the production of muscle proteins, recovery from surgery and sports-related injuries, and is integral with the production of hormones, enzymes, and antibodies in the body. Lysine is also an important component of collagen and has been shown to lower high serum triglyceride levels. It is found in high concentrations in lentils and leafy greens.

Methionine, or (S)-2-amino-4-(methylsulfanyl)-butanoic acid, is an essential amino acid that is, along with cysteine, the only sulfur-bearing amino acid. Methionine is synthesized in plants and microorganisms from aspartic acid and cysteine. Methoionine acts as the “chain initiator” for protein sequences. Methionine is a powerful antioxidant and helps to prevent buildup of fat deposits in the liver and arteries.

Phenylalanine, or 2-amino-3-phenyl-propanoic acid, is an essential amino acid that can take the form of a white powder at room temperature. Phenylalanine is found in most protein-rich foods. Phenylalanine is used by the brain to produce norepinephrine, a chemical used to transmit signals between nerve cells in the brain, and is attributed to blocking pain receptors. People that suffer from the disorder “phenylketonuria” cannot absorb phenylalanine in their normal metabolic processes.

Proline, or (S)-pyrrolidine-2-carboxylic acid, is a non-essential amino acid with a slightly different molecular structure than the other nineteen amino acids: it primary amine group is locked up in a pyrrolidine ring instead of being free. Proline is an important stabilization component of collagen and helps to reinforce joins and tendons. Proline is pronounced in heart tissues. Proline, in conjunction with Vitamin C, promotes healthy connective tissues—a shortage of proline would give the affect of scurvy.

Serine, or (S)-2-amino-3-hydroxypropanoic acid, is a non-essential amino acid that participates in the biosynthesis of purines, pyrimidines, cysteine, and other metabolites. Serine plays a role in the catalysis of enzymes in acting as the activity site for the enzyme. Serine acts as a metabolizer for fats and fatty acids, it is a component of the myelin sheaths that protect nerve cells, and aids in the production of immunoglobulins and antibodies.

Threonine, or (2S,3R)-2-amino-3-hydroxybutanoic acid, is an essential amino acid that helps regulate a proper protein balance in the body. Threonine also aids in the formation of collegen, elastin, and tooth enamel. Threonine also has a lipotropic function when combined with aspartic acid and methionine. Foods high in threonine are cottage cheese, poultry, fish, meat, lentils, and sesame seeds.

Tryptophan , or (S)-2-amino-3-(1H-indol-3-yl)-propionic acid, is an essential amino acid that is a precursor for serotonin (a neutrotransmitter), melatonin (a neurohormone), and niacin. Tryptophan also is credited as having anti-depressant properties and a pain receptor blocker. Tryptophan also enhances the release of growth hormone and aids in weight control.

Tyrosine, or (S)-2-amino-3-(4-hydroxy-phenyl)-propanoic acid, is a non-essential amino acid that is the precursor to throxine and triiodothyronine, two thyroid hormones, as well as hormones from the adrenal and pituitary glands. Tyrosine is also involved with the skin pigment melanin as well as involved with the production of adrenaline, dopamine, norepinephrine, and epinephrine.

Valine, or (S)-2-amino-3-methyl-butanoic acid, is an essential amino acid and a BCAA. Valine, is needed for muscle metabolism and coordination, tissue repair, and maintaining a proper nitrogen balance in the body. It may also be used by muscles as an energy source. Nutritional sources of valine include cottage cheese, fish, poultry, peanuts, sesame seeds, and lentils.

In some embodiments, a source of carbohydrates is included in the liquid composition. A source of carbohydrates is provided to help the consumer of the composition, who is typically using the composition during exercise or soon thereafter, quickly digest and obtain carbohydrate-based energy to continue exercising or recover from exercise to perform other activities. In some embodiments, a form of carbohydrate is used that the body can absorb and metabolize quickly to release energy relatively soon after consumption. In some embodiments, a form of carbohydrate may be provided that is more complex in chemical structure and, therefore, more difficult to digest, thereby causing a slower release of energy as the complex carbohydrate is metabolically broken down to simpler components. In some embodiments, different types of carbohydrates that are metabolized at different rates are combined.

There are various sources of carbohydrates that may be used to produce compositions for human consumption. For example, carbohydrates that are based upon simple sugars such as mono- and disaccharides include glucose, maltodextrin, polydextrose, fructose (crystalline or liquid), dextrose, sucrose (crystalline or liquid), lactose, and so on, are relatively easy to digest and metabolize by the body, thereby releasing their energy quickly. Other sources of carbohydrates—such as complex or polymeric carbohydrates—take more time to digest; however, in doing so they release their energy over a longer period of time. Examples of complex or polysaccharides include glucose polymers, pectin, corn starch, corn syrup solids; gums such as xanthum, guar, carrageenan, Arabic, locust bean, and tragacanth; rice syrup, rice starch, sorghum syrup, tapioca syrup, tapioca starch, and barley syrup, and so on. For example, a small glucose polymer molecule where n=4 (where n=number of repeating units between endcap units) might take the body some time to fully metabolize, but once it does it will have yielded an amount of energy equivalent to metabolizing 24 molecules of dextrose, but notably over a longer period of time.

It will be understood by those having skill in the art that the carbohydrate or blend of carbohydrates selected depends on the various attributes of the ingredients. For example, different carbohydrates provide energy more or less quickly and a judicious selection of same can contribute to balancing the delivery of carbohydrate calories to the consumer so that the energy provided is meted out over a period of time. In some embodiments, a combination of simpler and more complex carbohydrates may provide a carbohydrate metabolism profile and energy release that is suitable to a consumer using the product during exercise and recovering from exercise over a number of hours. In combining simple and complex carbohydrates, the body typically prefers to digest simple carbohydrates first to quickly release the caloric energy to immediate use. The body will then focus on the more complex carbohydrates to release energy over a period of time it takes to fully metabolize the more complex chemical structure. Such a combination may be used to help a consumer use the product immediately, thereby getting a quick energy release to permit continued exercise and to assist in recovery during the next 24-48 hours wherein the more complex carbohydrates are metabolized and provide energy.

The aqueous phase, typically comprising 88% by weight of the composition, is water. In some embodiments, the water is purified by de-ionization. In some embodiments, the water is purified by distillation. In some embodiments, the water is purified by reverse osmosis.

In various embodiments, the composition may contain other ingredients in order to provide a final product that is acceptable for consumer consumption and provides for bodily nutritional requirements during and after physical exertion. In some embodiments, flavoring materials such as natural and artificial flavors may be used to present, change, or enhance an overall flavor. In some embodiments, ingredients may be added that act as a delivery means for supplying aqueous electrolytes. In some embodiments, colorants are added to change the appearance of the composition. Examples include certified color additives and natural color additives like beet powder, beta carotene, and caramel. In some embodiments, additives are included to make the composition overall isotonic, wherein the goal is to produce an overall composition that is approximately 0.9 weight percent (9 g/L) salt in aqueous solution. In some embodiments, preservatives, emulsifiers, and stabilizers are added to preserve the composition and prevent decomposition over time and temperature exposure and maintain physical appearance. In some embodiments, carbohydrates and amino acids as previously described will contribute functionality in various areas such as flavoring, coloring, emulsification, stabilization, and preservation.

In some embodiments, ingredients are used to change the natural taste of a composition. For example, in various embodiments sodium chloride (NaCl) may be added to a composition to enhance the flavors that are present. Sodium chloride is well known in the art for enhancing the taste and flavor of foods by its aqueous electrolytic activity. For another example, in various embodiments sodium citrate (Na₃C₆H₅O₇) may be added to add a saline, mildly tart, flavor. In another example, in various embodiments citric acid, or 2-hydroxypropane-1,2,3-tricarboxylic acid, may be added to provide a slightly sour taste, act as a masking agent for stronger, more “off” or pungent-flavored composition ingredients by oxidation, and act as a pH buffer. As can be appreciated, a variety of artificial and natural flavorings, such as spices and extracts, can be added to present, change, or enhance an overall flavor for the composition to create a desirable impression and taste to a consumer.

During physical exertion, the body may begin to lose lighter metals and non-metals that provide the means for important biological functions and regulation of metabolisms in the body. In some embodiments, ingredients are added to enhance the delivery of important elements to the body of a consumer during and after exercise. For example, in some embodiments, sodium chloride may be added to provide a source for solubilized sodium, an element lost during physical exertion through sweat and an important electrolyte in the body. In some embodiments sodium citrate may be added to provide free sodium to the body. Citrate salts of various metals are known to deliver biologically available metals in many dietary supplements. In another example, in various embodiments, monopotassium phosphate (KH₂PO₄) may be added to provide a source for both potassium, an element important in nerve function and in influencing osmotic balance between cells and the interstitiual fluid, and phosphorous, an important component found in most cellular activities. Monopotassium phosphate may also act as a pH buffer for the composition.

In various embodiments, in addition to the amino acids, the carbohydrates, and the aforementioned ingredients, the compositions may include other ingredients such as, for example, vitamins, minerals, other non-essential amino acids, pH modifiers, stabilizers, emulsifiers, preservatives, and other functional ingredients, in a manner well known to those skilled in the art to achieve the desired nutritional goal of the composition.

For the purposes of this patent application, the terms “about” and “approximately” mean ±1% of the stated value.

The compositions may be prepared using various techniques. For example, in the case of a liquid composition, generally speaking, dry ingredients will be premixed with treated water and added to the liquid components. The resultant homogenized mixture may be bottled, sterilized, and cooled.

### EXAMPLE 1

A fruit-flavored liquid composition provides a source of energy in the form of carbohydrates and amino acids to repair and rebuild muscle fiber, with about the following weight percentages:

TABLE 1
Example 1 Composition Mixture

| | Wt % |
|---|---|
| Amino Acid Powder | 3.58 |
| Sodium Citrate | 0.37 |
| Sodium Chloride | 0.28 |
| Potassium Phosphate | 0.04 |
| Flavoring | 1.00 |
| Sucrose Syrup | 5.64 |
| Glucose/Fructose Syrup | 5.64 |
| Coloring | Neg. |
| Purified Water | 83.45 |
| Total: | 100.00 |

wherein the amino acid powder is about as follows:

TABLE 2
Example 1 Amino Acid Powder Composition

| | Wt % |
|---|---|
| Alanine | 9.45 |
| Arginine | 8.77 |
| Aspartate | 6.84 |
| Cystine | 0.09 |
| Glutamine | 10.98 |
| Glycine | 25.06 |
| Histidine | 1.22 |
| Isoleucine | 1.62 |
| Leucine | 3.33 |
| Lysine | 4.41 |
| Methionine | 1.71 |
| Phenylalanine | 2.11 |
| Proline | 14.12 |
| Serine | 3.91 |
| Threonin | 2.21 |
| Tryptophan | 0.05 |
| Tyrosine | 1.67 |
| Valine | 2.51 |
| Total | 100.06 |

It is understood to one of ordinary skill in the art that the sum total of each composition is 100.00% and that slight errors due to mathematical rounding do not detract from the accuracy or amounts given in any example.

What is claimed is:

1. A composition comprising:
a plurality of amino acids;
wherein the plurality of amino acids contains at least one essential amino acid and at least one non-essential amino acid; and
wherein the plurality of amino acids contains at least one branch-chain amino acid;
a source of carbohydrates;
purified water; and
wherein the plurality of amino acids comprises about 1 wt % of the composition.

2. The composition of claim 1, wherein the at least one essential amino acid is selected from the group consisting of isolucine, leucine, lysine, methionine, phenylalanine, threonine, tryptophan, valine, histidine, arginine, and mixtures thereof.

3. The composition of claim 1, wherein the at least one non-essential amino acid is selected from the group consisting of alanine, asparagine, aspartate, cysteine, glutamate, glutamine, glycine, proline, serine, tyrosine, and mixtures thereof.

4. The composition of claim 1, wherein the at least one branch-chain amino acid is selected from the group consisting of leucine, isoleucine, and valine.

5. The composition of claim 1, wherein the source of carbohydrates is selected from the group consisting essentially of mono-, di-, and polysaccharides, and mixtures thereof.

6. The composition of claim 5, wherein the source of carbohydrates is a mixture of saccharides that are metabolized at different rates.

7. The composition of claim 1, wherein the composition comprises a flavorant.

8. The composition of claim 7, wherein the favorant is a masking agent.

9. The composition of claim 8, wherein the flavorant is citrus acid.

10. The composition of claim 1, wherein the composition comprises an electrolyte.

11. The composition of claim 10, wherein the electrolyte is sodium chloride.

12. The composition of claim 10, wherein the electrolyte is monopotassium phosphate.

13. The composition of claim 10, wherein the electrolyte is a citrate salt.

14. A composition comprising:
a plurality of amino acids;
a sodium citrate;
sodium chloride;
potassium phosphate;
a flavoring;
a source of carbohydrates; and
purified water.

15. The composition of claim 14, wherein:
the plurality of amino acids is about 1 wt %;
the sodium citrate is about 0.37 wt %;
sodium chloride is about 0.28 wt %;
potassium phosphate is about 0.04 wt %;
the flavoring is about 1.00 wt %; and
the source of carbohydrates is about 11.28 wt %.

16. The composition of claim 14, wherein the plurality of amino acids are selected from the group consisting of isolucine, leucine, lysine, methionine, phenylalanine, threonine, tryptophan, valine, histidine, arginine, alanine, asparagine, aspartate, cysteine, glutamate, glutamine, glycine, proline, serine, and tyrosine.

17. The composition of claim 16, wherein the plurality of amino acids comprises:
alanine of about 9.45 wt %;
arginine of about 8.77 wt %;
aspartate of about 6.84 wt %;
cystine of about 0.09 wt %;
glutamine of about 10.98 wt %;
glycine of about 25.06 wt %;
histidine of about 1.22 wt %;
isoleucine of about 1.62 wt %;
leucine of about 3.33 wt %;
lysine of about 4.41 wt %;
methionine of about 1.71 wt %;
phenylalanine of about 2.11 wt %;
proline of about 14.12 wt %;
serine of about 3.91 wt %;
threonin of about 2.21 wt %;
tryptophan of about 0.05 wt %;
tyrosine of about 1.67 wt %; and
valine.

18. The composition of claim 14, wherein the source of carbohydrates comprises sucrose syrup, fructose syrup, and glucose syrup.

19. The composition of claim 14, comprising a colorant.
